(19) United States
(12) Patent Application Publication
(10) Pub. No.: US 2008/0003266 A1
(43) Pub. Date: Jan. 3, 2008

(54) Composition Comprising Statins

(76) Inventors: Christiaan Mitchael Beindorff, Vlaardingen (NL); Willem Maarten Meijer, Vlaardingen (NL); Henricus Otto Franciscus Molhuizen, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/579,027

(22) PCT Filed: Mar. 23, 2005
(86) PCT No.: PCT/EP05/03247

§ 371 Date: Oct. 27, 2006

(30) Foreign Application Priority Data
Apr. 28, 2004 (EP) 04076293.2
Aug. 3, 2004 (EP) 04077219.6

(51) Int. Cl. A61K 31/351; A61K 47/00; A61P 9/00; C12P 17/06

(52) U.S. Cl. 424/439; 435/125; 514/460

(57) ABSTRACT

A composition comprising statin wherein the composition is a flour comprising less than 10 wt. % of fat. Use of the composition comprising statin in the preparation a food product. Process for the preparation of the composition comprising statin.

### TECHNICAL FIELD

The present invention relates a composition comprising statin. The invention further relates to the use of the composition for the preparation of a food product and to a process for the preparation of the composition.

### BACKGROUND OF THE INVENTION

Cardiovascular disease is a leading cause of morbidity and mortality, particularly in the United States and in Western European countries and is emerging in developing countries. According to the American Heart Association, the major risk factors for heart disease are:

age (65 years and older), male gender, heredity, smoking, high blood-cholesterol levels (hypercholesteremia), high blood pressure, physical inactivity, diabetes, and obesity.

Obviously, some of these risk factors, such as age, gender, heredity, or diabetes, cannot be changed. However, it is possible to make lifestyle modifications which may decrease the risk of factors such as smoking, elevated blood-cholesterol, elevated blood pressure, physical inactivity, or obesity.

Elevated low-density lipoprotein cholesterol (hereafter “LDL-cholesterol”) is directly related to an increased risk of coronary heart disease.

Statins are compounds that are known to have a lowering effect on levels of LDL-cholesterol in the human blood. Statins inhibit the hydroxymethylglutaryl coenzyme A (HMG-CoA) reductase, the rate-determining step in the cholesterol biosynthesis.

Scientific research has confirmed the healthy properties of statins especially with respect to LDL blood-cholesterol and triglyceride levels lowering activities, both in animals and in humans (Li et al., Nutrition Research 18, 71-81 (1998); Heber et al., Am. J. Clin. Nutr. 69, 231-236 (1999)).

The presence of statins in food consumed by humans is associated with a lower level of LDL-cholesterol and lower risk of coronary heart disease.

People are considered obese when their BMI is over 30. The BMI number is calculated as a person's weight divided by that person's height. People with two or more risk factors for heart disease are at even higher risk for heart disease, when combined with a high BMI. In addition, obesity increases mortality of coronary heart disease 2-4 fold.

It is therefore imperative to have a healthy weight. Long term modification in diet to achieve and maintain weight loss is essential. Bread, potatoes, pasta, rice fruit and vegetable are considered to be the basis of a healthy diet. It is generally acknowledged that dietary fats should be kept to a minimum.

It would be beneficial to have a product that manages more than one risk-factors.

Compositions comprising statins for the manufacture of food products are known. WO02/64809 describes a process for the preparation of statins by fermentation and food products comprising one or more statins. It describes the extraction of statins from fermented soya beans with organic solvents (ethanol and acetonitril) and the use of the extract for the preparation of magarine and spreads. The drawback of this process is that the yield of the statins is rather low (0.0545 g statin/kg (ethanol extract) and 0.0978 g statin/kg (acetonitril extract)).

In WO02/063976, fermented soy material comprising statin is used for the preparation of the food products. This fermented soy material comprises about 20 wt. % oil. The high fat-content makes these kinds of statin containing material unsuitable for use in a low-fat diet.

### SUMMARY OF INVENTION

It is an object of the present invention to provide a composition comprising statins that can be used for the manufacture of food products. It is another object of the invention to provide a composition comprising statins that can be used for the manufacture of low-fat food products. Furthermore another object of the invention is to provide a low-fat food product comprising statins. Yet another object of the invention is to provide a process for the preparation of the composition comprising statins.

Surprisingly one or more of the above objects is obtained by a composition comprising statin wherein the composition is a flour comprising less than 10 wt. % of fat.

### DETAILED DESCRIPTION OF THE INVENTION

The present invention relates to a composition comprising statin characterised in that the composition is a flour comprising less than 10 wt. % of fat. Preferably the flour comprises less then 5 wt. % of fat, preferably less then 1 wt. % of fat or even more preferably essentially no fat.

For the purpose of this invention flour is defined as a fine powder obtained by grinding a starchy plant material. Flour may be made from starchy plant materials including barley, buckwheat, chickpeas, lima beans, oats, peanuts, potatoes, soybeans, rice, and rye.

The composition according to the present invention comprises statins. Statins are defined as substances having the structural formula, presented in FIG. 1. In this structural formula, R1 and R2 can be any group. Preferred statins are those which are given in FIG. 1.

The amounts of statins given will be expressed, in wt. % or weight parts per million (ppm), mg/kg or mg/g, relative to the total weight of the food product, unless otherwise indicated.

The amounts of statins given herein are the sum of the amounts of individual statins, as e.g. determined by high performance liquid chromatography (HPLC) or LC-MS, unless otherwise indicated.

Preferably the composition according to the present invention comprises at least 0.01 mg/g statin, more preferably 0.1 mg/g statin. Even more preferred the flour of the invention comprises at least 1 mg/g statin.

One embodiment of the present invention relates to the use of the composition of the present invention in the preparation a food product.

Several food products may be prepared according to the invention, for example, spreads, soups, pasta, noodles, ice-cream, sauces, dressing, snacks, cereals, beverages, bread, biscuits, muffins, other bakery products, sweets, bars, chocolate, dairy products, dietetic products such as slimming products or meal replacers etc. In particular bakery products such as pancakes, muffins, and bread may be prepared according to the invention.

The food product according to the invention, in particular bakery products, has a low fat content. Preferably the food product has less than 10 wt. % of fat, more preferably less than 5 wt. %.

The food product according to the invention preferably comprises statins in an amount sufficient to obtain a blood LDL-cholesterol lowering effect if the food product is used according to the common needs of the consumer.

The preferred intake of statin per day is herein 5-40 mg/day, more preferably 5-20 mg/day, even more preferably 8-15 mg/day. Furthermore, the intake of statin per day is preferably 1-5 mg/day, more preferably 1-2.5 mg/day.

The skilled person will be able to adjust the percentage of statins in the food product to obtain the desired blood cholesterol lowering effect. The percentages will depend on the type of food product, since the food products are used in different serving sizes. Moreover the pattern in a food product is consumed (servings per day and distribution over days) is dependent on the food product.

Preferably the food product comprises 0.1-10 mg statin per serving, more preferably 1-5 mg statin per serving and even more preferably 1-2.5 mg statin per serving.

Another embodiment of the present invention relates to a process for the preparation of composition comprising statin wherein the composition is a flour comprising less than 10 wt. % of fat and the process comprises the steps of fermenting a substrate with a statin producing fungus, grounding the substrate, extracting the substrate, and recovering the extracted substrate, whereby the extracted substrate results in a flour comprising statin with less than 10 wt. % of fat. The substrate can be grounded before or after the fermentation. Preferably the grounding is performed after the fermentation.

The fermented substrate may be extracted with any extracting agent provided that the extracting agent extracts the fat from the substrate. For use in food applications, the extraction of fat from fermented soybeans can be performed with a number of organic solvents: hexane, acetone, ethyl acetate and ethanol or mixtures of these. Preferably the extracting agent extracts the fat from the substrate so that the extracted substrates comprises less than 10 wt. % fat, more preferably less than 5 wt. % fat, and even more preferably less than 1 wt. % fat. In addition, the preferred extracting agent leaves an effective amount of statin in the substrate. An effective amount of statins stands for an amount sufficient to be effective in cholesterol-lowering. Preferably the extracted substrate comprises at least 0.01 mg statin per gram extracted substrate, more preferably at least 0.1 mg statin per gram extracted substrate, even more preferred at least 1 mg statin per gram extracted substrate. Preferably the extracting agent is food grade. Hexane and super-critical CO₂ are preferred extracting agents.

The substrate may vary depending on whether it may be fermented with a statin producing fungus. In addition, the substrates preferably have a high content of starchy material. Suitable substrates that may be used are soybeans, barley, buckwheat, chickpeas, lima beans, oats, peanuts, potatoes, rice, and rye.

Especially suitable substrate may be soybeans. Soybean flour may be used for a variety of products such as cookies, pastries, sweet goods, salty snacks e.g. nacho chips, prepared meals, meat replacer, pasta, pancakes, muffins, bread and bread products. Furthermore, soybean flour comprises protein, fibers and phytoestrogen, saponins, polyphenols such as isoflavones, preferably in the non-glycosylated form. All of which have been shown to have a beneficial effect on the blood-cholesterol level and the triglycerides level in the blood.

Soy protein content in a food product may be measured according to the method described in Agater et al., J. Sci. Food Agric. (1986), 37(3), 317-31.

Polyphenols include flavenoids, which include isoflavones. The polyphenols include isoflavones, stilbenes, lignans, coumestans and resorcyclic acid lactones. Examples of isoflavones are genistein, daidzein, equol, glycitein, biochanin A, coumestrol, maitaresinol, formononetin, O-desmethylengolesin, enterolactone and enterodiol. Preferred isoflavones according to the invention are genistein and daidzein and glycitein, which are present in soybeans.

Saponins are herein derived as β-D-glucopyranosiduronic acid derivates. Examples of saponins are Soya sapogenol A,B,C,D and E, Soyasaponin I, II and III, as described in Lebensmittel Lexikon, B.Behr's Verlag GmbH & Co. Hamburg, Bd.2, L-Z-3, 1993, pages 550-552.

Preferably the food product according to the invention comprises statin and non-glycosylated isoflavone. In soy beans and soy materials derived from soy, isoflavones are present substantially in the glycosylated form. Typically about 5 wt. % of the isoflavones is present in the non-glycosylated form. The most important glycosylated isoflavones are genistin, daidzin and glycetin. The non-glycosylated forms are respectively genistein, daidzein and glycetein. Genistein, daidzein and glycetein have been reported to have advantageous health effects, including estrogenic and antioxidant properties.

Due to the fermentation according to the invention the glycosylated isoflavones are converted into the corresponding non-glycosylated isoflavones, which are more benificial. For instance, the amount of genistein and daidzein is increased in the fermented soy compared to the non-fermented soy.

The isoflavone concentration may be measured according to the HPLC method described in Franke A. A., et al. (1998): HPLC analysis of isoflavonoids and other phenolic agents from foods and human fluids; Proceed. Soc. Exp. Biol. Med; 217 (3), 274-280.

It has been shown that statins can be produced by a variety of filamentous fungi, including Monascus, Aspergillus, Penicillium, Pleurotus, Pythium, Hypomyces, Paelicilomyces, Eupenicillium, and Doratomyces.

Preferably the fungus is chosen from the group consisting of Monascus fungi and more preferably from the group consisting of Monascus ruber fungi.

As a food product, rice fermented with a red Monascus fungus (red rice) has been known and used for hundreds of years in China. Red rice was used and still is used in wine making, as a food-colouring agent and as drug in traditional Chinese medicine. Most red rice available on the market contains no statins or statins in very low amounts. The Food and Drug Administration has concluded that red yeast rice available in the market does not contain significant amounts of lovastatin (FDA, Docket No. 97-0441, Final Decision).

WO 99/23996 describes a composition for treating elevated serum cholesterol and/or triglycerides comprising a red rice product containing at least 0.05% lovastatin by weight.

Red rice powder capsules are sold as dietary supplements under the name of Cholestin by the firm Pharmanex. Pharmanex also sells a Cholestin bar containing red yeast rice (Monascus purperus went).

Red rice has an intensive red colour. Whereas the intensive red colour of red rice is an advantage when it is used as colouring agent, it is a disadvantage when it is used in food products. Due to the intense red colour of red-rice products, the foods prepared from red rice are coloured, depending on the amount of red-rice product added to the food product yellow, orange or red. The higher the amount of red rice added to the food, the more intense is the red colour of the food product.

In some food products the red colouring is undesirable. In particular in the western world, consumers are reluctant to use products of which the colour has changed from that they are used to. For example bread, pancakes, muffins, spreads, including margarine, butter, low fat spreads or salad oils are considered unacceptable by customers, when the colour of such a product is orange or red. However, at the same time these type of products have been found by us to be excellent vehicles of the daily intake of amounts of statins sufficient to obtain a blood LDL-cholesterol lowering effect.

When colours are classified, they can be broken down into the three primary elements. One is the Hue (colour) the other is Value (brightness) and the third is Chroma (Saturation like vivid colours or dull colours).

To enable anyone to tell anyone else exactly what colour they are talking about a common numerical code is used. This numerical code used is L*a*b*. When a colour is expressed in this system, Value becomes L*, while Hue and Chroma are expressed as a* and b* respectively. The L*a*b* may be measured with a UV 1601 spectofotometer of Shimatzu.

Preferably the food product has a Hue a* value of less than 20, preferably less than 10, most preferably less than 0.

A preferred is Monascus strain is Monascus ruber F125 M1-4. This strain when grown on soybeans doesn't have a red colouring.

Strain F125 M1-4 is deposited at the Centraal Bureau voor Schimmelculturen (CBS) as no. CBS 109269 on 23 Jan. 2001.

These deposits were made under the provisions of the Budapest Treaty on the International Recognition of the Deposit of Microorganisms for the Purpose of Patent Procedure and the Regulations thereunder (Budapest Treaty).

The fermentation may be carried out in a manner, which can be determined by the skilled person on the basis of the methods described in WO02/064809 and WO02/063976.

The fermentation temperature may be important. The temperature is preferably in the range of 10 to 37° C., more preferably 20 to 30° C.

Preferably during fermentation the medium is aerated, e.g. by stirring, shaking etc. Aeration may be carried out by blowing air through the fermentation medium. Preferably the air is wholly or partly saturated with water vapour in case solid state fermentation is used. This avoids drying out of the fermentation medium.

The levels of statins will depend on the fermentation time. The fermentation time is therefore dependent on the desired amount of statins. Preferred fermentation time is 1-50 days, more preferably 15-40 days, most preferably 20-30 days (See WO02/064809 and WO02/063976).

### DESCRIPTION OF THE DRAWING

FIG. 1 shows a schematic representation of the structure of different statins.

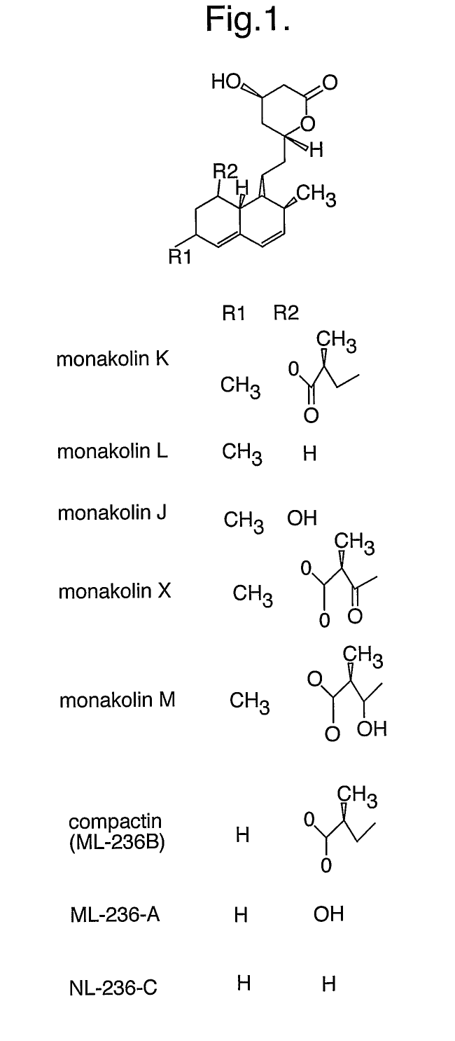

FIG. 2: shows a schematic representation of the experimental set-up for supercritical carbon dioxide extraction.

### EXAMPLES

Determination of Lovastatins Using HPLC

Samples were prepared by adding an extraction mixture, containing acetonitrile, water and phosphoric acid (1:1:0.05, v/v/v) to the material in a ratio of 5:1. Usually 5 g of material was extracted with 25 ml of extraction mixture. Samples were incubated for 1 hour at room temperature and then homogenised using an Ulta-Turrax. After homogenisation the samples were incubated overnight at room temperature on a roller bench. The samples were centrifuged at 11.000 rpm for 10 minutes and the supernatants collected for HPLC analysis. The amount of lovastatin in the samples was determined by HPLC separation according to the method of Morovjan et al. J. chromatogr. A 763 (1997) 165-172.

The system consists of the Shimadzu SCL-10A system controller, CTO-10AS column oven, LC-10AT vp pump system, RID-10A refraction index detector, SPD-M10A diode array detector and SIL-10AD autoinjector. For the chromatographic determination of lovastatin a Waters NovaPak C18 (150×3.9 mm I.D., 4 μm) column was used operating at 25° C. The eluent was an acetonitrile, water and phosphoric acid (1:1:0.05 v/v/v) solution flowing at 1.5 ml/min. Runs were performed for 15 min. The detection was performed using a diode array detector from 190 nm up to 800 nm. The sum of the area of all peaks in the spectrum belonging to lovastatin is measured. Comparison to a standard (Mevinolin, Sigma) allows the calculation of a lovastatin content (expressed in mg/g analysed product).

### Example 1-8

### Preparation of Fermented Substrates

Inoculum Preparation

Monascus ruber F125 M1-4 (CBS 109269) was plated on VMA-agar plates and incubated at 30° C. for 3 days.

With a sterile scalpel, small squares were cut in the VMA-agar for the preparation of inoculates. With a sterile spatula, the blocks of agar were transferred to the liquid media. Malt water was used for pre-cultivation. Sterile flasks of 500 ml were filled with 300 ml medium. The flasks were incubated in an Innova 400 shaker at 25° C. for 2 days.

Fermentation Process

Substrates like soybeans, kidney bean, mung bean, lupine seeds, walnut, maize, oat and peanuts were soaked in tap water (50° C.) for 60 minutes. After soaking the substrates were rinsed with cold tap water. Subsequently the substrates were air-dried at ambient temperature for 180 minutes. The soaked and dried substrates were transferred to a shake-flask, approximately 50 g per flask. The shake flasks were sterilised by autoclaving (10 min at 120° C.), inoculated with 1 ml of a fully-grown Monascus culture and incubated for an appropriate time (2-6 weeks) at 25° C. Lovastatin production is monitored and when a sufficient level has been obtained, the flasks are pasteurised by placing the flasks in an incubator at 80° C. overnight after which the end product can be harvested. Table 1 gives an overview of the statin content of the end product after 3 weeks of fermentation.

TABLE 1
Amount of statin in end product after 3 weeks of fermentation

| Example | Substrate | Statin g/kg |
|---|---|---|
| 1 | Soy beans | 1.2 |
| 2 | Kidney beans | 1.2 |
| 3 | Mung beans | 1.2 |
| 4 | Lupine seeds | 1.5 |
| 5 | peanut | 0.4 |
| 6 | Walnut | 0.8 |
| 7 | Maize | 0.3 |
| 8 | Oat | 1.4 |

### Example 9

### Supercritical Extraction of Fermented Soybeans

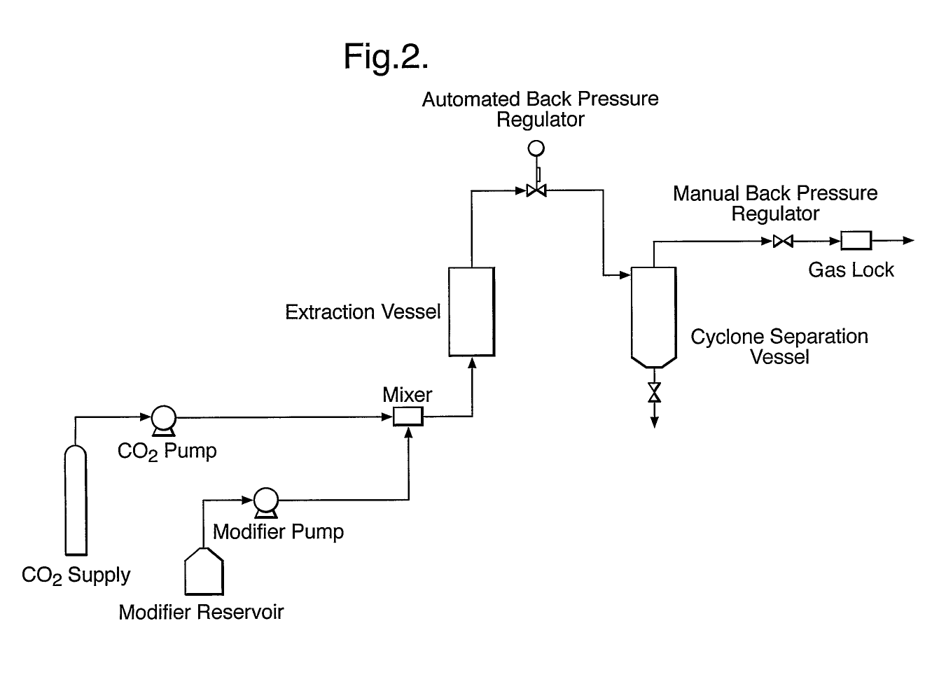

For the supercritical extraction of natural solid matrices, equipment and software of Thar Designs, USA was used. The experimental set-up is schematically depicted in FIG. 2. The CO₂ pump is capable of compressing liquid carbon dioxide to a pressure up to 600 bars at a constant flow-rate. In a static mixer, a polarity modifier may be mixed with the liquid carbon dioxide. In a pre-heater (not depicted) the carbon dioxide was heated to reach supercritical conditions before entering the extraction vessel.

In the extraction vessel, which was heated with a double wall heating mantle, the supercritical carbon dioxide was passed over the solid matrix for extraction.

Downstream of the extraction vessel, the supercritical carbon dioxide was expanded over an automated backpressure regulator. The backpressure regulator was coupled to a feedback control unit to control the pressure in the system. The carbon dioxide was separated from the extracted material (liquid/solid) in a cyclone separation system. The carbon dioxide left the cyclone at the top, while the extracted material remained in the cyclone. The liquids extracted from the solid matrix were recovered during the experiment by opening the valve at the bottom of the cyclone.

The carbon dioxide gas was further expanded over a further backpressure regulator, which was operated manually. A gas clock downstream of the backpressure regulator registers how much gas has been put through the system, before the carbon dioxide leaves the system at ambient pressure.

The process equipment is designed to operate at the following conditions:

TABLE 2
Process operation conditions supercritical extraction set-up

| Parameter | Range | |
|---|---|---|
| Flow rate of liquid carbondioxide | 5-150 | g/min |
| Pressure | up to 600 bar | |
| Temperature | 20-95° | C. |
| Extractor volume | 500 | mL |
| Cyclone volume | 200 | mL |

| | |
|---|---|
| Matrix to be extracted | solid |

The fermented soybeans were ground prior to extraction in a water-cooled universal mill (type M20, IKA, Germany) until a fine powder was obtained.

The amount of sample used for extraction was 100 gram of ground fermented soybeans. The fermented and ground soybeans were put in the extraction vessel and the remaining volume was filled with small glass beads (2 mm diameter). The flow rate of liquid carbondioxide was 20 g·min⁻¹. In the attached cyclone separation vessel, the extracted oil was collected. The total extraction time was 2 hours.

After the extraction, the extraction vessel was opened and a dry free-flowing soybean flour powder was obtained. The added glass beads were removed by sieving. An aliquot of the soybean flour was analysed for the amount of Lovastatin. Both in the starting material as well as in the extracted material the amount protein, moisture fat and ash were determined. An example of the analytical results is presented in Table 3.

TABLE 3
Composition of grounded fermented soybeans before
and after supercritical fluid extraction.

Grounded fermented
soybeans

| | Before | After |
|---|---|---|
| | extraction | extraction |
| Protein (wt. %) | 43.5 | 51.6 |
| Fat (wt. %) | 19.4 | 3.3 |
| Moisture (wt. %) | 6.0 | 3.6 |
| Ash (wt. %) | 6.0 | 6.9 |
| Lovastatin (mg/g) | 0.61 | 1.04 |

### Example 10

### Soxhlet Extraction With Hexane of Fermented Soybeans

### Comperative Example A

### Soxhlet Extraction With Ethanol of Fermented Soybeans

150 g of grounded fermented soybeans was put in an extraction thimble. 800 ml of organic solvent was added and the extraction was performed for 6 hours. After the extraction, the extraction thimble was taken out of the extraction equipment. The extraction thimble was emptied and the obtained soy powder was dried on air until constant weight. An aliquot was taken for the determination of the lovastatin concentration. Both in the starting material as well as in the extracted material the amount of ash, protein fat and moisture were determined. The analytical results are shown in Table 4.

TABLE 4
Result of Soxhlet extraction of Monascus fermented soybeans.

Grounded fermented soybeans

After extraction

| | |
|---|---|
| Ex. 10 | Comp. Ex. A |

| | |
|---|---|
| Before | Solvent |

| | extraction | Hexane | Ethanol |
|---|---|---|---|
| Protein (wt. %) | 43.4 | 55.2 | 45.4 |
| Fat (wt. %) | 17.4 | 4.0 | 14.4 |
| Moisture (wt. %) | 1.6 | 2.9 | 5.8 |
| Ash (wt. %) | 5.7 | 7.8 | 6.6 |
| Lovastatin (mg/g) | 0.94 | 1.42 | 0.66 |

Both with supercritical fluid extraction as well as with hexane extraction soybean fat is efficiently removed resulting in a defatted soy flour(fat content <5 wt. % ). In addition, in both cases there still remain sufficient Lovastatin present in the defatted soy flour. Ethanol is less suitable as it does not remove the fat as efficient as hexane or supercritical fluid and also leaves less statin in the extracted flour.

### Example 11

### Preparation of Low-Fat Oat-Pancakes with Banana and Orange

Serving size: 65 g

Formulation:

| Ingredient | (wt. %) |
|---|---|
| Rolled oats | 6.3 |
| Unbleached flour | 8.0 |
| soy flour with 1 mg/g statins, 4% fat | 4 |
| Baking powder | 1.7 |
| Plain soy milk | 45 |
| Banana (thinly sliced) | 16 |
| Orange (thinly sliced) | 19 |
| Total | 100 |

Preparation:

In a large bowl, combine the rolled oats, unbleached flour, soy flour and baking powder. Add the soymilk, and blend with a few swift strokes. Fold in the banana and orange slices.

Pour 0.25 cup (65 g) of the batter onto a hot non-sticky griddle or pan. Cook for about 2 min or until bubbles appear at the surface. Flip the pancake and cook for another minute or until heated through.

The pancakes can be served with e.g. maple syrup, apple sauce or a fruit spread.

Serving size 65 g, per serving: 2.6 mg of statins,

### Example 12

### Preparation of Low-Fat Raisin Bread

Formulation:

| Ingredient | (wt. %) |
|---|---|
| Bread flour | 42.5 |
| soy flour with 1 mg/g statins, 4% fat | 5.4 |
| Oatmeal | 4 |
| Salt | 0.7 |
| Active dried yeast | 1.4 |
| Sugar | 3 |
| Plain soy milk | 34 |
| Raisins | 5 |
| Orange (skins) (dried and rehydrated with sugar syrup) | 4 |
| Total | 100 |

Preparation:

Mix the ingredients thoroughly in a dough mixer. Proof the dough. Allow the dough to rise for 30-60 min at a temperature of 20° C. Put the risen dough in a bread tin. Bake the bread at 190° C. for 30-60 min.

Serving size: 40 g, amount of statins per serving 2.2 mg.

### Example 13

### Preparation of Low-Fat Orange-Banana Muffins

Formulation:

| Ingredient | (wt. %) |
|---|---|
| soy flour with 1 mg/g statins, 4% fat | 8.7 |
| All purpose flour | 8.9 |
| Whole wheat flour | 8.6 |
| Baking soda | 0.7 |
| All spice powder | 0.5 |
| Dried and rehydrated orange skin cubes | 5.4 |
| Honey | 21.4 |
| Raisins | 9.3 |
| Orange juice concentrate | 3.6 |
| Vanilla | 0.9 |
| Egg whites | 14.3 |
| Bananas | 17.7 |
| Total | 100 |

Preparation:

Combine dry ingredients in bowl. Mix orange juice, vanilla, banana, honey and half of the egg whites in a bowl on low speed until blended. Add dry ingredients and mix for a few seconds, just until moistened and no flour can be detected.

Beat the remaining egg whites and stir into the above mixture. Add raisins. Fill paper cupcake liners in muffin tins and fill cups to within 0.3 inch of top. Bake at 180° C. for 10-24 min until you can touch the muffin lightly on top and the imprint does not remain.

Serving size: 75 g, amount statins per serving: 6.5 mg.

The flour with statins may be party replaced by whole wheat flour and/or all purpose flour, this allows for easy variation of the statin content.

### Example 14

### Preparation of Low-Fat Pasta

| Ingredient | (wt. %) |
|---|---|
| Ibis Flour (NBS/14% PROT) | 74.65 |
| Tapioca starch pre cooked 20% (Nat. Starch) | 8.50 |
| Palm oil | 1.13 |
| soy flour with 1 mg/g statins, 4% fat | 1.13 |
| Natrium Alginate -Manucol Dmb (Kelco) | 0.06 |
| Titanium dioxide | 0.01 |
| CaCl2 | 0.01 |
| water | 14.51 |
| total | 100 |

The ingredients were mixed to form the pasta. The pasta was then formed with a pasta maker into long noodle-like threads. The pasta was cooked for 5 minutes. Serving size 80 g, amount statins per serving: 1 mg. The consumption of 80 g pasta would result in an estimated blood cholesterol lowering of 5%.

1. Composition comprising statin characterised in that the composition is a flour comprising less than 10 wt. % of fat.

2. Composition according to claim 1 comprising less than 5 wt. % of fat.

3. Composition according to claim 2 comprising less than 1 wt. % of fat.

4. Composition according to claim 1 comprising at least 0.01 mg/g statin.

5. Composition according to claim 4 comprising at least 0.1 mg/g statin.

6. Composition according to claim 5 comprising at least 1 mg/g statin.

7. Use of a composition according to claim 1 in the preparation a food product.

8. Use according to claim 7 wherein the food product is a bakery product.

9. A food product comprising statin characterised in that it comprises less than 10 wt. % of fat.

10. A food product according to claim 9 having a Hue a* value of less than 20, preferably less than 10, most preferably less than 0.

11. A food product according to claim 9 selected from the group consisting of muffins, bread, pancake, and pasta.

12. Process for the preparation of composition comprising, statin wherein the composition is a flour comprising less than 10 wt. % of fat, and wherein the process comprises the steps of
fermenting a substrate with a statin producing fungus,
grounding the substrate,
extracting the substrate, and
recovering the extracted substrate,
whereby the extracted substrate results in a flour comprising statin with less than 10 wt. % of fat.

13. Process according to claim 12 wherein the substrate is extracted with hexane or super-critical CO₂.

14. Process according to claim 12 wherein the substrate are soybeans.

15. Process according to claim 12 wherein the statin producing fungus is a Monascus fungus.
